(19) United States
(12) Patent Application Publication
(10) Pub. No.: US 2018/0313752 A1
(43) Pub. Date: Nov. 1, 2018

(54) SYSTEM UTILIZING A NARROW COLLIMATED BEAM OF OPTICAL RADIATION TO DETECT THE PRESENCE OF A HYDROCARBON GAS

(71) Applicant: Pixel Velocity, Inc., Ann Arbor, MI (US)

(72) Inventors: David McCubbrey, Ann Arbor, MI (US); Larry Peterson, Ann Arbor, MI (US); Eric Larson, Ann Arbor, MI (US)

(21) Appl. No.: 15/768,168

(22) PCT Filed: Oct. 11, 2016
(86) PCT No.: PCT/US16/56360

§ 371 Date: Apr. 13, 2018

Related U.S. Application Data
(60) Provisional application No. 62/242,058, filed on Oct. 15, 2015.

(51) Int. Cl. G01N 21/39; G01N 21/3504; G01M 3/38; G01N 33/00

(52) U.S. Cl. CPC G01N 21/39; G01N 33/0047; G01M 3/38; G01N 21/3504

(57) ABSTRACT

A device for detecting the presence of a hydrocarbon gas includes a an optical assembly and a digital analyzer. The optical assembly includes a laser source and an optical receiver, which may be an infrared optical receiver. The laser source is configured to output a beam towards a target, while the optical receiver is configured to receive a reflected portion of the beam reflected from the target. The optical receiver outputs a waveform to the digital analyzer, the waveform being based on the reflected portion of the beam reflected from the target that is receiver by the optical receiver. The digital analyzer is configured to receive the waveform from the optical receiver and determine if a hydrocarbon gas is present between the optical assembly and the target. The beam outputted by laser source of the optical assembly may be a collimated beam.

### CROSS REFERENCE TO RELATED APPLICATION

This application claims priority to U.S. Provisional Application No. 62/242,058 filed on Oct. 15, 2015 all of which are herein incorporated by reference in their entirety.

### BACKGROUND

### 1. Field of the Invention

The present invention generally relates to systems to detect the presence of a hydrocarbon gas.

### 2. Description of Related Art

Hydrocarbon gases, as well as other gases including normal atmospheric constituent gasses (oxygen, nitrogen, carbon dioxide, carbon monoxide, ammonia, water vapor, etc.), each have specific radiation absorption spectra unique to the gas molecular structure, occurring in various portions of the electromagnetic spectrum. Detection of small proportions of a specific gas of interest, specifically each specific hydrocarbon gaseous compound such as methane, is often of concern because leakage of such a gas may constitute a toxic threat and environmental pollutant that creates safety, health and other adverse effects when present to any significant degree.

Leakage also creates economic losses due to product loss and fines from violation of regulations concerning avoidance of environmental pollution. In order to sensitively detect and remediate clouds of methane (or any other such specific gas of concern) it is desirable to be able to measure the absorption of infrared radiation, within the specific wavelength interval where that gas has relatively high absorption, through the entire depth of any such gas cloud that may be present. The absorption of the emitted infrared radiation as it passes through the gas cloud depends upon the gas cloud density at all points along the path of that radiation, both as the outgoing radiation passes through the cloud and as any portion of the radiation beam energy is reflected back through the cloud to the instrument detector at the point of origin.

Current systems for detecting the presence of hydrocarbon gas clouds may include forward-looking infrared sensors, such as passive thermal cameras. These passive thermal cameras do not have a high level of wavelength selectivity so that the signal received by the passive thermal cameras includes multiple infrared radiation noise sources from the ambient natural and man-made environment. These systems may produce excessive false alarms due to the energy received at a very large number of infrared wavelengths due to numerous atmospheric and other natural, and man-made infrared sources, which energy cannot be separated from the particular absorption spectrum of a specific hydrocarbon gas of interest. Furthermore, these systems depend on the presence of favorable environmental conditions such as a warm background between the thermal camera and the gas.

In addition to these drawbacks, these systems are also costly to implement due in part to the inherent expense of high-resolution infrared cameras, which may require thermal cooling. In some cases, some solutions involve the use of a human operator to visually examine lower resolution images produced by such thermal cameras in conjunction with making rounds to periodically inspect potential leakage sources at various sites. While this solution may work in theory, the solution is costly due to high labor costs and stresses the attention span of the human operator.

As a result, prior art solutions are incapable of reliably detecting hydrocarbon densities well below the explosion threshold, thus creating serious safety and damage threats as well as economic loss.

### SUMMARY

A device for detecting the presence of a hydrocarbon gas includes an optical assembly and a digital analyzer. The optical assembly includes a laser source and an optical receiver, which may be an infrared optical receiver. The laser source is configured to output a beam towards a target, while the optical receiver is configured to receive a reflected portion of the beam reflected from the target. The optical receiver outputs a waveform to the digital analyzer, the waveform being based on the reflected portion of the beam reflected from the target that is receiver by the optical receiver. The digital analyzer is configured to receive the waveform from the optical receiver and determine if a hydrocarbon gas is present between the optical assembly and the target. The beam outputted by laser source of the optical assembly may be a collimated beam.

The device embodies a relatively economical sensing and measurement instrument which reliably detects and quantitatively determines the hydrocarbon content within multiple small volumes throughout dynamically changing hydrocarbon gas clouds, by measuring the absorption of controlled laser illumination by the instrument at one infrared wavelength, or a certain very narrow range of infrared wavelengths, characteristics of the particular hydrocarbon gas of interest, such narrow beam of illumination being sequentially directed by the instrument control unit at selected elevation and azimuth angles from the instrument into its 3-dimensional volumetric field-of-regard in which no such cloud, or one or more such clouds, may be present.

Further objects, features, and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1 illustrates a block diagram of a system having a device for detecting the presence of a hydrocarbon gas;

FIG. 2 illustrates an example of a waveform of the absorption spectrum of methane near to the wavelength of a helium-neon laser; and

FIG. 3 illustrates an example of a waveform showing the limitation of prior art solutions.

### DETAILED DESCRIPTION

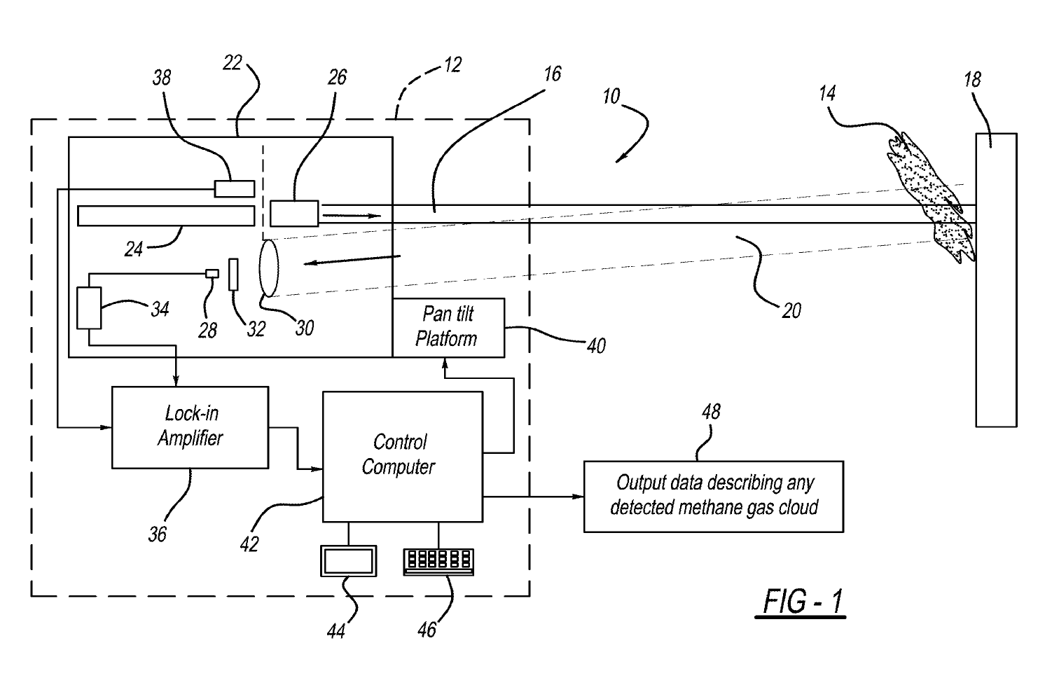

Referring to FIG. 1, a block diagram of a system 10 having a device 12 for detecting the presence of a hydrocarbon gas, such as a hydrocarbon gas cloud 14 is shown. Broadly, the device 12 emits a beam 16, which may be a collimated beam, towards a target 18. The target 18 may be any solid surface that is capable of reflecting portions of the beam back to the device 12. For example, the target, may be a heat exchanger and/or a non-specular solid object or surface of any kind.

As such, the reflected beam 20 is reflected off the surface of the target 18 and back towards the device 12. If a hydrocarbon gas cloud 14 is present, the reflected beam 20 will have characteristics indicative of the presence of a hydrocarbon gas cloud 14.

In order to produce the beam 16, the device 12 may include an optical assembly 22. The optical assembly 22 may include a laser source 24. The laser source 24 may be a helium-neon laser. The laser 24 outputs a beam to a beam expander 26. The beam expander 26 functions to collimate the beam 16 and direct the beam 16 towards the target 18.

The optical assembly 22 may also include an optical receiver 28. The optical receiver 28 may be an infrared optical receiver. More specifically, the optical receiver 28 may be a mercury-cadmium-telluride (MCT) infrared sensor that is optionally cooled to minimize noise such as by employing a thermoelectric cooler. The reflected beam 20 may be focused on the optical receiver 28 using one or more receiver optics, so as to focus the reflected beam 20 to the optical receiver 28. For example, receiver optics 30 and 32 may be present so as to focus the reflected beam 20 on the optical receiver 28.

The optical receiver 28 is configured to output a waveform based on the reflected beam 20 received by the optical receiver 28. This waveform may then be provided to different analog or digital systems to receive analog and/or digital filtering. For example, the waveform may be provided to a preamplifier 34, and it may be later provided to a lock-in amplifier 36.

The lock-in amplifier 36 may be a type of amplifier that can extract a signal with a known carrier wave from an extremely noisy environment. Depending on the dynamic reserve of the instrument, signals up to 1 million times smaller than noise components, potentially fairly close by in frequency, can still be reliably detected. It is essentially a homodyne detector followed by low pass filter that is often adjustable in cutoff frequency and filter order. Whereas traditional lock-in amplifiers use analog frequency mixers and RC filters for the demodulation, state of the art instruments have both steps implemented by fast digital signal processing for example on a field programmable gate array.

Sine and cosine demodulation may be performed simultaneously, which is sometimes also referred to as dual phase demodulation. This allows the extraction of the in-phase and the quadrature component that can then be transferred into polar coordinates, i.e. amplitude and phase, or further processed as real and imaginary part of a complex number.

Because the beam 16 outputted to the target 18 should be synchronized with the optical receiver 28, an optical chopper 38 may be utilized. It should be understood that the optical receiver 28 should be synchronized to the laser source 24 when sampling the reflected beam 20. The chopper 38 synchronizes the timing of laser emissions and the sampling of the detector amplified signal.

The device 12 may also include a pan-tilt platform 40. The pan-tilt platform 40 allows the device 12 to be positioned such that the beam 16 from the device 12 can be focused on different areas of the target 18.

The device 16 may also include a digital analyzer 42. The digital analyzer 42 may be configured to receive the waveform from the optical receiver 28. As stated before, the waveform may go through some form of digital and/or analog filtering. The digital analyzer 42 may be configured to receive the waveform from the optical receiver and determine if a hydrocarbon gas is present between the optical assembly 22 and the target 18. Further, the digital analyzer 42 may be configured to receive the waveform from the optical receiver and determine if the hydrocarbon gas is at least one of the following ethane, propane, butane, or any other hydrocarbon gasses having a molecular carbon-hydrogen bond.

The digital analyzer 42 may be in communication with the pan-tilt platform 40 so as to control the pan-tilt of the device 12. Additionally, the digital analyzer 42 may have an output device 44 and/or an input device 46. The output device 44 may be a display or printer or any suitable output device. The input device may be a keyboard and/or mouse, or any suitable input device. It should also be understood that the digital analyzer 42 may be separate from the device 12. For example, the digital analyzer 42 may be a general-purpose computer capable of receiving from the device 12 waveforms generated by the optical receiver 28.

In order to accurately measure the overall size, shape and density profile of any gas cloud 14 that is present, the beam 16 must be narrow enough to sequentially measure the integrated path absorption along multiple narrowly focused radiation azimuth-elevation pointing angles. Furthermore, to sample the gas cloud 14 with a sampling density that is independent of the range from the device 12 to the gas cloud 14, a collimated beam 16 of radiation from the device 12 will be desirable, such as may be created by use of conventional beam expander optics 26. The diameter of the radiated infrared beam 16 in each instance may be selected after considering such factors as the field-of-regard geographic size and the minimum size of the gas cloud 12 that is desired to be detectable. The overall size and shape and density profile of any gas cloud 14 of interest is obtained by combining the measured integrated path absorption along many different radiation directions that are accurately spaced in elevation and azimuth across the extent of the gas cloud 14 (laterally to the emitted radiation beam projection angle).

In one operational implementation and system 10 set-up strategy (of many that may be chosen), the system 10 can be programmed to initiate a complete repetitive operational cycle by searching (i.e., radiating its narrow infrared beam) in sequence, at a variety of azimuth/elevation angle combinations selected to accomplish a relatively coarse sampling of areas of interest in the system's 10 overall field-of-view. In such a strategy, when the integrated path absorption measured at one of these selected pointing angles indicates the potential presence of a gas cloud 14 of interest, then the device 12 may be controlled, by any conventional pan-tilt device 40 via the digital analyzer 42, to proceed to radiate its beam to a sequence of closely surrounding pointing angles so as to map out the lateral extent of the potential gas cloud 14 and develop a profile of the integrated path absorption across the entire lateral extent of the cloud. Other strategies obviously may be selected according to the a priori knowledge available for any specific site and its unique gas leak detection operational requirements, including but not limited to spatial resolution, response time and sensitivity.

The radiated infrared beam 16 employed in the disclosed invention can provide, under various specific operational conditions, two (2) to three (3) decimal orders of magnitude greater sensitivity for detecting the specific cloud compound for which the specific instrument's radiation wavelength has been optimized. For example, reliable methane detection may be achieved for methane cloud concentrations of 0.005%, which compares favorably to the commonly referenced methane explosion danger level of 4.4%.

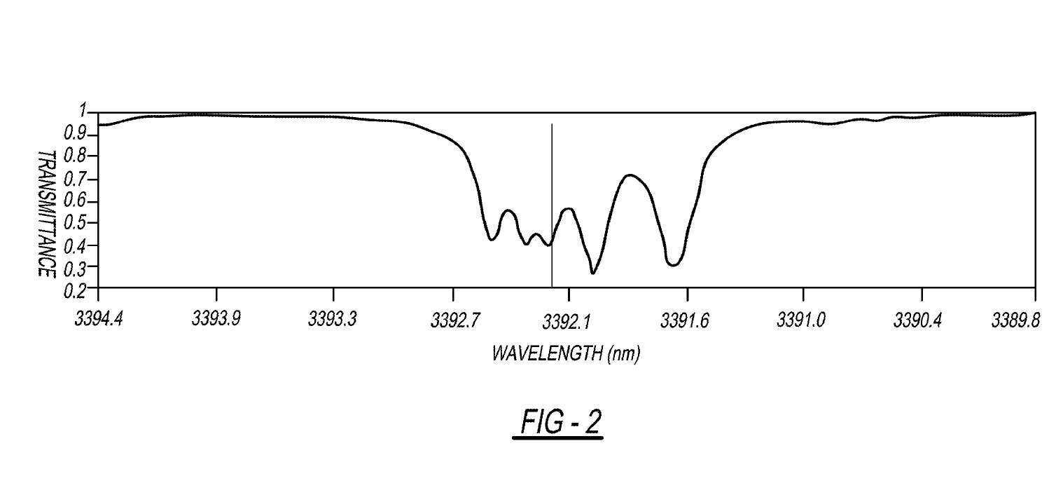

FIG. 2 illustrates a waveform in units of transmittance, an example of the absorption spectrum of methane near to the wavelength of the laser source 24 configured with conventional mirror components, well known in the art, to suppress visible red laser emissions in favor of lasing to produce the stronger HeNe infrared emission at the single wavelength of 3392.2348 nanometers. The specific example case illustrated is for the device 12 observing 0.001 atmosphere-meters of methane through a standard atmosphere. (The standard atmosphere includes all normal atmospheric component gases, but water vapor is the only atmospheric component having absorption in the thermal infrared region.)

The receiver 28, being provided with conventional optical filters, pre-amplification 34 and lock-in amplifier 36, then detects reflected energy in only that one wavelength and chopping frequency from the currently energized spot in the field of regard, so as to detect the two relative magnitudes of the received radiation at that wavelength, which will be substantially different (such as 50% different for the case illustrated) if the example level of methane is present along that current line of emission and sight.

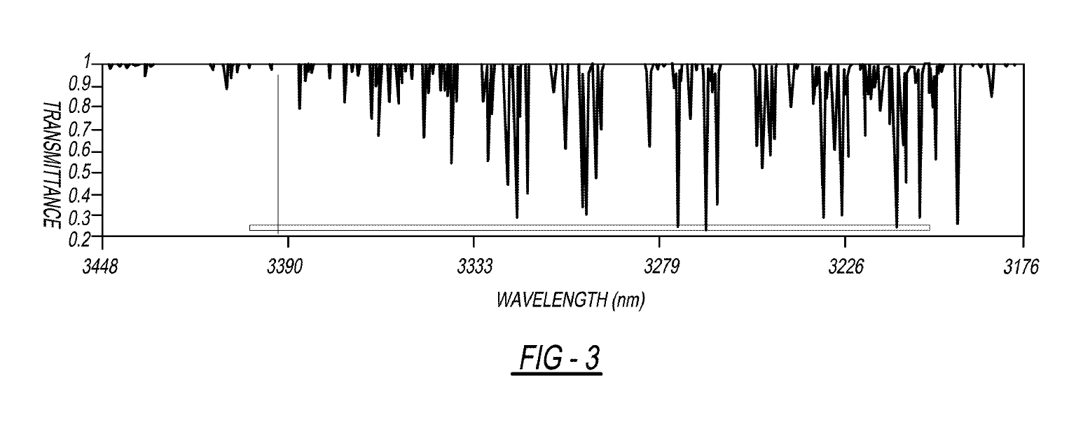

FIG. 3 illustrates a waveform that indicates the limitations of prior art in comparison to the invention disclosed here. Note that the horizontal axis of the waveform of FIG. 3 extends over 272 nanometers whereas waveform of FIG. 2 extends over only 4.6 nanometers. The prior art (specifically passive thermal cameras) senses, averages, and cannot separate, the received radiation across a much wider portion of the infrared spectrum indicated by the wide red horizontal line in that waveform of FIG. 3.

In that broad portion of the infrared spectrum, the average atmospheric absorption when the example level of methane is present in the same composition as in FIG. 2 is only approximately 2%, as indicated by the many spaces between absorption peaks shown in FIG. 3. In this typical case, the disclosed invention will have an approximate 25-to-1 sensitivity advantage over the prior art.

In an alternative embodiment, dedicated hardware implementations, such as application specific integrated circuits, programmable logic arrays and other hardware devices, can be constructed to implement one or more of the methods described herein. Applications that may include the apparatus and systems of various embodiments can broadly include a variety of electronic and computer systems. One or more embodiments described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

In accordance with various embodiments of the present disclosure, the methods described herein may be implemented by software programs executable by a computer system. Further, in an exemplary, non-limited embodiment, implementations can include distributed processing, component/object distributed processing, and parallel processing. Alternatively, virtual computer system processing can be constructed to implement one or more of the methods or functionality as described herein.

Further, the methods described herein may be embodied in a computer-readable medium. The term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the methods or operations disclosed herein.

As a person skilled in the art will readily appreciate, the above description is meant as an illustration of the principles of this invention. This description is not intended to limit the scope or application of this invention in that the invention is susceptible to modification, variation, and change, without departing from the spirit of this invention, as defined in the following claims.

1. A device for detecting the presence of a hydrocarbon gas, the device comprising:
an optical assembly having a laser source and an optical receiver;
a digital analyzer in communication with the optical receiver;
the laser source is configured to output a beam towards a target;
the optical receiver is configured to receive a reflected portion of the beam reflected from the target;
the optical receiver is configured to output a waveform to the digital analyzer, the waveform being based on a reflected portion of the beam reflected from the target that is received by the optical receiver; and
the digital analyzer configured to receive the waveform from the optical receiver and determine if a hydrocarbon gas is present between the optical assembly and the target.

2. The device of claim 1, wherein the beam outputted by the optical assembly is a collimated beam.

3. The device of claim 2, further comprising a beam expander configured to receive the beam from the laser source and output the collimated beam.

4. The device of claim 1, wherein the output of the laser beam by the laser source is synchronized with optical receiver, so that the emission of the laser beam by the laser source is synchronized with sampling by the optical receiver.

5. The device of claim 4, further comprising an optical chopper for synchronizing the emission of the laser beam by the laser source with sampling by the optical receiver.

6. The device of claim 1, further comprising a lock-in amplifier configured to receive the waveform and output a modified waveform the to the digital analyzer

7. The device of claim 1, further comprising the target, wherein the target is a heat exchanger.

8. The device of claim 1, further comprising the target, wherein the target is a non-specular solid object or surface of any kind.

9. The device of claim 1, wherein the optical receiver further comprises an infrared receiver.

10. The device of claim 7, wherein the infrared receiver is a mercury-cadmium-telluride infrared sensor.

11. The device of claim 1, wherein the digital analyzer configured to receive the waveform from the optical receiver and determine if the hydrocarbon gas includes a molecular carbon-hydrogen bond.

12. The device of claim 9, the hydrocarbon gas is at least one of the following: ethane, propane, or butane.

13. The device of claim 1, further comprising a pan-tilt platform, wherein the pan-tilt platform is configured to position the device such that the beam from the device be focused on different areas of the target

14. A device for detecting the presence of a hydrocarbon gas, the device comprising:
an optical assembly having a laser source and an optical receiver;
a digital analyzer in communication with the optical receiver;
the laser source is configured to output a beam towards a beam expander;
the beam expander configured to receive the beam from the laser source and output a collimated beam towards a target;
the optical receiver is configured to receive a reflected portion of the beam reflected from the target;
the optical receiver is configured to output a waveform to the digital analyzer, the waveform being based on a reflected portion of the beam reflected from the target that is receiver by the optical receiver;
the digital analyzer configured to receive the waveform from the optical receiver and determine if a hydrocarbon gas is present between the optical assembly and the target,
wherein the beam outputted by the optical assembly is a collimated beam, and
wherein the output of the laser beam by the laser source is synchronized with optical receiver, so that the emission of the laser beam by the laser source is synchronized with sampling by the optical receiver.

15. The device of claim 14, further comprising an optical chopper for synchronizing the emission of the laser beam by the laser source with sampling by the optical receiver.

16. The device of claim 14, further comprising a lock-in amplifier configured to receive the waveform and output a modified waveform the to the digital analyzer

17. The device of claim 14, wherein the digital analyzer configured to receive the waveform from the optical receiver and determine if the hydrocarbon gas includes a molecular carbon-hydrogen bond.

18. The device of claim 18, further comprising a pan-tilt platform, wherein the pan-tilt platform is configured to position the device such that the beam from the device be focused on different areas of the target.
